(19) United States
(12) Patent Application Publication
(10) Pub. No.: US 2010/0261784 A1
(43) Pub. Date: Oct. 14, 2010

(54) AGENT FOR REDUCING INTESTINAL TOXIC BACTERIUM AND FOOD OR PHARMACEUTICAL PREPARATION COMPRISING THE SAME

(75) Inventor: Fumitaka Ueda, Ashigarakami-gun (JP)

Correspondence Address: SUGHRUE-265550, 2100 PENNSYLVANIA AVE. NW, WASHINGTON, DC, 20037-3213, US

(73) Assignee: FUJIFILM CORPORATION, Tokyo (JP)

(21) Appl. No.: 12/746,615

(22) PCT Filed: Dec. 7, 2007
(86) PCT No.: PCT/JP2008/072592

§ 371 Date: Jun. 7, 2010

(30) Foreign Application Priority Data
Dec. 7, 2007 (JP) 2007-217132

(51) Int. Cl. A61K 31/353; A61Q 19/00; A61P 3/10; A61P 29/00; A61P 15/00; A61P 17/00; A61K 31/05; A61P 1/00

(52) U.S. Cl. 514/456; 514/731

(57) ABSTRACT

An agent for reducing an intestinal toxic bacterium is provided, the agent including: a pulverized product or extract of a plant of the genus Salacia.

### TECHNICAL FIELD

This invention relates to an agent for reducing an intestinal toxic bacterium or toxic substance, which comprises a pulverized product or extract of a plant of the genus Salacia and a food or pharmaceutical preparation containing its components.

### BACKGROUND ART

The root and trunk of a plant of the genus Salacia have been used as a natural drug by a traditional medical science, aayurveda, in India and Sri Lanka. It has been handed down in Sri Lanka that the root skin of Salacia reticulata is effective in treating rheumatism, gonorrhea and a skin disease and is also used in the treatment of initial stage diabetes mellitus. In India, a root of Salacia oblonga is used in similar treatments, and it is said that Salacia chinensis is also used in the treatment of diabetes mellitus (FOOD Style 21, vol. 6, no. 5, pp. 72-78).

Thus, it has been handed down that plants of the genus Salacia are effective in the prevention and early stage treatment of diabetes mellitus. In recent years, it has been reported that a plant of the genus Salacia has the action to suppress increase of blood sugar level, and its action mechanism is the sugar absorption suppressing action based on the α-glucosidase activity inhibition (FOOD Style 21, vol. 6, no. 5, pp. 72-78).

In addition, certain compounds which are contained in the extraction components of the genus Salacia and have the action to inhibit α-glucosidase activity (Japanese Patent No. 3030008, JP-A-2004-323420 and JP-A-2000-86653), and their application examples as anti-diabetic agents based on the α-glucosidase activity inhibitory action (JP-A-9-301882 and Japanese Patent No. 3261090), have been known.

Regarding effects of the pulverized products and extracts of plants of the genus Salacia on the stomach and intestines, there is a report stating that these are effective as the motor accelerator of digestive organ systems (Japanese Patent No. 3771789), but there is no description on toxic bacteria and toxic substances in the intestines.

Also, there is a report stating that these can improve intestinal environment by their concomitant use with lactic acid bacilli and bifidobacterium (JP-A-2007-31345), but since separation from the effects of lactic acid bacilli and bifidobacterium originally having the intestinal environment improving action has not been made, the effect of Salacia is not clear. In addition, since there is no description also on the toxic bacteria and toxic substances in the intestines, specific effect of Salacia is not clear.

### DISCLOSURE OF THE INVENTION

According to this report, an agent for reducing an intestinal toxic bacterium is provided. In addition, by revealing efficacy of the plants of the genus Salacia in the intestines, which has not so far been known clearly, a food article and pharmaceutical preparation to which the efficacy is applied are provided. Particularly, by finding new effects to lower intestinal pH, to reduce intestinal ammonia concentration, to reduce intestinal putrefaction product concentration, to accelerate intestinal bifidobacterium propagation and to improve chapped skin, these improving measures are provided.

Though most of the effects of Salacia so far reported are as described in the above, the present inventors have intensively examined this time on the influences of the ingestion of Salacia upon the intestines, chapped skin and physical conditions and found as a result that Salacia reduces toxic bacteria in the intestines, reduces ammonia and the like toxic substances in the intestines and increases bifidobacterium (Bifidobacterium) and the like good bacteria.

The invention consists of the following constructions.

(1) An agent for reducing an intestinal toxic bacterium, which comprises:

a pulverized product or extract of a plant of the genus Salacia.

(2) The agent for reducing an intestinal toxic bacterium as described in (1) above, which shows an activity as a sucrase 50% inhibition concentration (IC₅₀ value) of 50 μg/ml or more and 1,000 μg/ml or less.

(3) The agent for reducing an intestinal toxic bacterium as described in (1) or (2) above, which further comprises:

from 1 to 50% by mass of catechin.

(4) The agent for reducing an intestinal toxic bacterium as described in any one of (1) to (3) above, which further comprises:

from 2 to 80% by mass of polyphenols having lipase activity inhibitory effect.

(5) The agent for reducing an intestinal toxic bacterium as described in (1) above,

wherein the intestinal toxic bacterium to be reduced is a bacterium of the genus Enterobacter or a bacterium of the genus Clostridium.

(6) The agent for reducing an intestinal toxic bacterium as described in any one of (1) to (4) above, which is an intestinal pH lowering agent.

(7) The agent for reducing an intestinal toxic bacterium as described in any one of (1) to (4) above, which is an intestinal ammonia concentration reducing agent.

(8) The agent for reducing an intestinal toxic bacterium as described in any one of (1) to (4) above, which is an intestinal putrefaction product concentration reducing agent.

(9) The agent for reducing an intestinal toxic bacterium as described in (8) above,

wherein the putrefaction product is indole or skatole.

(10) The agent for reducing an intestinal toxic bacterium as described in any one of (1) to (4) above, which is an intestinal bifidobacterium propagation accelerator.

(11) The agent for reducing an intestinal toxic bacterium as described in any one of (1) to (4) above, which is a chapped skin improving agent.

(12) A food or drink or a food or drink material, which comprises:

the agent for reducing an intestinal toxic bacterium as described in any one of (1) to (4) above.

(13) A tablet or hard capsule filling type food article or pharmaceutical preparation, which comprises:

the agent for reducing an intestinal toxic bacterium as described in any one of (1) to (4) above.

### BEST MODE FOR CARRYING OUT THE INVENTION

### Salacia

The agent of the invention for reducing an intestinal toxic bacterium contains a pulverized product or extract of a plant of the genus Salacia. As the plant of the genus Salacia, Salacia reticulata, Salacia prinoides, Salacia oblonga and the like plants of the family Celastraceae, genus Salacia, can be used. Particularly, Salacia reticulata, which is also called kothalahimbutu, can be suitably used.

According to the invention, preferably an extract or pulverized product from at least one species selected from the group consisting of a trunk, a root skin and leaf of a plant of the genus Salacia can be used.

It is desirable that the leaf is used as a small piece or powder by pulverizing it. In addition, it can also be ingested as such.

The root skin and trunk can be used as powders. In addition, these can also be used for the extraction of an extract. The term extract as used herein means an extract of a plant of the genus Salacia. When used in the extraction of an extract, these may be used as such, or the extract can be extracted after making into small pieces or powders by pulverizing them.

According to the invention, the extract of a plant of the genus Salacia may be any one of the filtrate after extraction as such, or its concentrated or diluted state or in the form of its dried powder, or a mixture thereof.

The dry extract powder of the aforementioned extract can be used as such when used or by dissolving in an appropriate solvent. The aforementioned solvent may be any substance, with the proviso that it is a solvent which can be used at the time of extraction and does not exert a bad influence upon the human body even when it remained in the drug or foodstuff after preparation, and water, an alcohol or a hydrous alcohol is preferably used. More preferably, hot water or ethanol or hydrous ethanol is used. Regarding the alcohol concentration of the aforementioned hydrous alcohol, those which have a concentration of from 30 to 90% by mass, preferably from 40 to 70% by mass, may be used. (In this specification, mass ratio is equal to weight ratio.) As the drying method, spray drying, freeze drying and the like can be exemplified, though not limited thereto.

It is preferable that the pulverized powder or powder-extracted extract powder of the root skin or trunk shows a weight loss on drying of 10% or less, more preferably a weight loss on drying of 8% or less, by the weight loss on drying test of The Pharmacopoeia of Japan.

In addition, the extract or pulverized product of a plant of the genus Salacia can also be used in the form of a paste or powder by concentrating and drying the same. When the extract is made into a paste or powder by concentrating and drying it, a freeze drying method, a spray drying method and the like are used, though not limited thereto. The extract of a plant of the genus Salacia made into a paste or powder can be ingested as such, or a dried extract powder of the extract of a plant of the genus Salacia may be made into a food article, by adding to and mixing with a material containing water, tea, coffee, juice, alcohol and the like drinks, a cake and the like general food and the like as an inclusion composition, in an amount of 0.01% by mass or more of the inclusion composition. It can also be used for an external use.

Particularly, the drinks are used as container-packed drinks. Since the appearance of a container-packed drink shows a large change in color tone when preserved for a prolonged period of time, it becomes unfit as goods. In the drinks, coloration gradually advances and the color tone is changed with the lapse of time.

By adding ascorbic acid, sodium ascorbate or the like antioxidant for the purpose of maintaining the color tone, it can exert further improved effect. Blending amount of the antioxidant is from about 0.03 to about 1.2% by mass, preferably from about 0.04 to about 1.0% by mass, more preferably from about 0.05 to about 0.8% by mass, based on the inclusion component.

<Other contents>

It is desirable that the agent of the invention for reducing an intestinal toxic bacterium contains flavonoid in addition to the extract or pulverized product of a plant of the genus Salacia.

Flavonoid is a general term for the pigment components distributing in all plant organs, which is contained mainly in fruits and vegetables and is present particularly in the form of glycosides in skins of green and white vegetables and citrus fruits.

According to the invention, the flavonoid is a general term for the pigment components broadly distributing in plants, and particularly, it means flavan derivatives frequently contained in vegetables and fruits.

As the flavonoid, flavonols, isoflavones and catechins are preferable. Flavonols are known as polyphenols.

Flavonoid is a substance ingested into the body, but is generally hard to be absorbed. However, since flavonoid is effective even at a small amount and is a strong antioxidant, it is known that it suppresses the activity of carcinogens and has blood circulation accelerating action and anti-thrombus action.

According to the invention, flavonoid can be obtained from tea, grape, onion and the like respective origins. In this case, the origins mean those which are extracted from at least a part of an organism. For example, the above-mentioned method for preparing an extract of a plant of the genus Salacia can be employed for the extraction, and the form of the extract can also be the same as described in the above; for example, it may be any one of the filtrate after extraction as such, or its concentrated or diluted state or in the form of its dried powder, or a mixture thereof.

The tea extract containing catechins is prepared from a tea plant which is an evergreen tree belonging to the family Theaceae. As the tea plant, both of the assamica cultivated in India, Sri Lanka and Southeast Asia and Camellia sinensis cultivated in China and Japan can be used. In general, water, an alcohol or a hydrous alcohol is preferably used in the extraction. More preferably, hot water or ethanol or hydrous ethanol is used as the extraction solvent. Regarding the alcohol concentration of the aforementioned hydrous alcohol, those which have a concentration of from 30 to 90% by mass, preferably from 40 to 70% by mass, may be used. As the drying method, spray drying, freeze drying and the like can be exemplified, though not limited thereto.

Polyphenol, catechins and the like antioxidants are contained in the tea extract. It is desirable that catechin, epicatechin, gallocatechin, epigallocatechin, catechin gallate, epicatechin gallate, gallocatechin gallate or epigallocatechin gallate is contained therein, and it is particularly desirable that epigallocatechin gallate is contained therein.

It is preferable that the agent of the invention for reducing an intestinal toxic bacterium contains said tea extract in an amount of from 0.1 to 40% by mass, more preferably from 0.5 to 35% by mass, particularly preferably from 1.0 to 30% by mass.

Also, the flavonols as one of the flavonoid eliminate active oxygen and thereby show anti-oxidation actions such as suppression of arteriosclerosis and improvement of blood circulation. Among the flavonols, resveratrol as one of the polyphenol has been drawing attention as an antioxidant. Resveratrol is constituted from the stilbene backbone and contained in a large amount in the rind of grapes so that it is also contained in red wine produced from grapes.

It is desirable that the invention comprises a grape extract or grape wine concentrate which contains said flavonols as a component.

It is preferable that the agent of the invention for reducing an intestinal toxic bacterium contains the grape extract in an amount of from 0.1 to 30% by mass, more preferably from 0.1 to 10% by mass.

It has been revealed that resveratrol has the actions to burn fat, to prevent a blood vessel system disease, arteriosclerosis, to exert anti-cancer action and to prevent shortening of DNA caused by cell division, and has the effect to prolong life of cells similar to the case of carrying out calorie control, so that it has the excellent effect as a material for preventing life style-related diseases.

The resveratrol content in the agent of the invention for reducing an internal toxic bacterium is preferably from 0.0001 to 5.00% by mass, more preferably from 0.001 to 2.00% by mass.

In addition, among the flavonols, quercetin as a polyphenol has been drawing attention as an antioxidant. Quercetin has the flavan structure and is contained in a large amount in onion skins.

Vitamin C absorption support, anti-oxidation action, immune action and the like physiological actions of quercetin have been reported, and it has been revealed that it is effective in suppressing fat absorption and it has been revealed also that it has the excellent effect as a material for preventing life style-related diseases.

The quercetin content in the agent of the invention for reducing an intestinal toxic bacterium is preferably from 0.001 to 15% by mass, more preferably from 0.05 to 10% by mass, further preferably from 0.1 to 5.0% by mass.

It is preferable that the agent of the invention for reducing an intestinal toxic bacterium contains catechin in an amount of from 1 to 50% by mass. As the catechin, a green tea-derived one or the like is particularly desirable.

In addition, it is preferable that the agent of the invention for reducing an intestinal toxic bacterium contains a polyphenol having lipase activity inhibitory effect in an amount of from 2 to 80% by mass. As the polyphenol having lipase activity inhibitory effect, those derived from Oolong tea, derived from grape, derived from apple, derived from Lychee, derived from pine bark, derived from kanka and the like are particularly desirable.

### <Performance>

Action to Reduce Intestinal Toxic Bacteria

As described in the above, the agent of the invention for reducing an intestinal toxic bacterium can reduce intestinal toxic bacteria through its ingestion, by containing a pulverized product or extract of a plant of the genus Salacia.

The intestinal toxic bacterium is particularly a toxic bacterium in the large intestine, and for example, a bacterium of the genus Clostridium, a bacterium of the genus Enterobacter and the like can be cited.

Intestinal Putrefaction Product

Also, reduction of an intestinal putrefaction product can be carried out by ingesting the agent of the invention for reducing an intestinal toxic bacterium. As the putrefaction product, indole and skatole can be particularly cited.

Other Actions

Also, propagation of bifidobacterium (a bacterium of the genus Bifidobacterium), so-called a good bacterium, can be accelerated by ingesting the agent of the invention for reducing an intestinal toxic bacterium.

In addition, optimization of intestinal pH, reduction of intestinal ammonia concentration, improvement of chapped skin and the like can be carried out by ingesting the agent of the invention for reducing an intestinal toxic bacterium.

It is desirable that the agent of the invention for reducing an intestinal toxic bacterium has a sucrase 50% inhibition concentration (IC₅₀ value) of 50 μg/ml or more and 1000 μg/ml or less. When the inhibition activity becomes smaller than this range, the glucose absorption inhibitory action from the digestive tracts becomes weak and the expected effects becomes slightly weak, and when it becomes large, a feeling of abdominal swelling and generation of gas becomes slightly strong. The sucrase 50% inhibition concentration is preferably 80 μg/ml or more and 600 μg/ml or less, more preferably 100 μg/ml or more and 450 μg/ml or less.

The sucrase 50% inhibition concentration (IC₅₀ value) is measured by the following method.

Measurement of Sucrase IC₅₀ Value

Preparation of sample solution: A 2 mg portion of a sample is weighed and put into a tube and thoroughly suspended in 2 ml of water added thereto, thereby preparing a sample solution having a concentration of 1 mg/ml. This is diluted with water to respective concentrations of 0, 50, 100, 250 and 500 μg/ml.

Preparation of substrate liquid: Sucrose is dissolved in 0.2 M maleate buffer (pH 6.0) to a sucrose concentration of 100 mM, and this is used as the substrate liquid.

Preparation of crude enzyme liquid: A 1 g portion of intestinal acetone powder rat (mfd. by SIGMA) is suspended in 10 ml of physiological saline and then centrifuged (3,000 rpm, 4° C., 5 min). The thus obtained supernatant is separated and used as the crude enzyme liquid.

A 400 μl portion of the substrate liquid is added to 500 μl of each of the aforementioned sample solution having respective concentrations and preliminarily heated at 37° C. for 5 minutes in a water bath. A 100 μl portion of the crude enzyme liquid is added to each of them and allowed to undergo the reaction at 37° C. for 60 minutes. After completion of the reaction, the reaction is terminated by deactivating the enzyme through heating at 95° C. for 2 minutes. Determination of concentration of the thus formed glucose is carried out using a commercially available kit for mutarotase glucose oxidase method (Glucose CII Test Wako, mfd. by Wako Pure Chemical Industries).

Preparation of blank: A 200 μl portion of the substrate liquid and 50 μl of the crude enzyme liquid are added to 250 μl of each of the aforementioned sample solution having respective concentrations and immediately heated at 95° C. for 2 minutes to effect thermal deactivation of the enzyme, to be used as blank data.

By preparing a calibration curve from the thus obtained values, the concentration which inhibits 50% of the enzyme activity (IC₅₀ value) is calculated.

<Shape>

The agent of the invention for reducing an intestinal toxic bacterium can be used as food and pharmaceutical preparations. In addition, the agent of the invention for reducing an intestinal toxic bacterium can take powder preparations, tablets, solutions, capsule preparations and the like various shapes.

According to the invention, in order to improve periodical discoloration of the extract powder extracted from the plant of the genus Salacia, it is desirable to contain 1% by mass or more of calcium carbonate or silicon dioxide as a desiccant in forming tablets or capsules.

Further, a low moisture absorption material, a moisture absorbent, an antioxidant and the like which are applicable as a foodstuff or food additive agent can be used. Preferably, cellulose, crystalline cellulose, cellulose powder, microcrystalline cellulose, lactose, an oligosaccharide, a sugar alcohol, trehalose, magnesium stearate, calcium stearate or the like is used as the low moisture absorption material. As the moisture absorbent, silicates, magnesium carbonate, a ferrocyanide, polysaccharides or the like are used. More preferably, crystalline cellulose, microcrystalline cellulose or lactose is used as the low moisture absorption material. As the antioxidant, ascorbic acid, sodium ascorbate or the like is used.

A compound necessary for forming into the powder, solid preparation or liquid preparation of the invention, and the like may be optionally contained. As examples of such a compound, erythritol, maltitol, hydroxypropylcellulose, kaolin, talc and the like can be cited.

According to the invention, conventionally known measures and conventionally known materials can be applied to the preparation for obtaining powder preparations, tablets or solutions, granulation of capsule inclusion matter for forming capsule preparations, capsulation, capsule material and the like.

The capsule preparation of the invention may be in a hard capsule, soft capsule, seamless capsule, microcapsule or the like shape, and is characterized in that the capsule shell is constructed by at least one or two or more species selected from pig skin gelatin, pig bone gelatin, fish gelatin and a natural hydrophilic polymer. A capsule shell of pig skin gelatin or fish gelatin is particularly desirable.

These capsule shells can be produced by a conventionally known method. In this case, the term “constructed by pig skin gelatin, pig bone gelatin, fish gelatin or a natural hydrophilic polymer” means that total amount of the pig skin gelatin, pig bone gelatin, fish gelatin or natural hydrophilic polymer is 30% by mass or more, preferably 40% by mass or more, more preferably 50% by mass or more, particularly preferably 60% by mass or more, based on the total mass of capsule shell.

In addition, in order to avoid its contact with air from the viewpoint of preventing oxidation, it is desirable to pack the above-mentioned composition in a packing bag or packing container.

### EXAMPLES

The following describes the invention based on examples, but the invention is not limited to the following examples.

### Example 1

Root and trunk parts of Salacia reticulata and Salacia oblonga were pulverized and then subjected to a hot water extraction step, and the thus obtained liquid was spray-dried to obtain a Salacia extract powder.

Powders of the following formulations were prepared using this Salacia extract powder, and their sucrase IC_{H} values were measured by the method described above.

Also, an Oolong tea powder was prepared by freeze-drying a commercially available Black Oolong teat (mfd. by Suntory). It was confirmed that this powder significantly inhibits swine pancreatic lipase.

In addition, Sunfenon 100s manufactured by Taiyo Kagaku (contains 55% by mass of catechin) was used as a green tea extract.

Using these, the formulation components shown in the following Table 1 were subjected to tablet making to prepare the samples 1 to 12.

TABLE 1
Salacia formulation example and sucrase IC₅₀ value

| | Salacia | | | | | |
|---|---|---|---|---|---|---|
| | extract | Green tea | Oolong tea | Crystalline | Sucrose | |
| | powder | extract | powder | cellulose | IC₅₀ value | Examples |
| Sample 1 | 0 mg | 0 mg | 0 mg | 250 mg | >2000 | Comparative |
| Sample 2 | 0 mg | 20 mg | 0 mg | 230 mg | 1020 | Comparative |
| Sample 3 | 0 mg | 0 mg | 100 mg | 150 mg | 3350 | Comparative |
| Sample 4 | 20 mg | 0 mg | 0 mg | 230 mg | 910 | Inventive |
| Sample 5 | 100 mg | 0 mg | 0 mg | 150 mg | 182 | Inventive |
| Sample 6 | 230 mg | 0 mg | 0 mg | 20 mg | 43 | Inventive |
| Sample 7 | 100 mg | 10 mg | 0 mg | 120 mg | 176 | Inventive |
| Sample 8 | 100 mg | 20 mg | 0 mg | 130 mg | 168 | Inventive |
| Sample 9 | 100 mg | 60 mg | 0 mg | 90 mg | 162 | Inventive |
| Sample 10 | 100 mg | 20 mg | 100 mg | 30 mg | 171 | Inventive |
| Sample 11 | 100 mg | 0 mg | 100 mg | 50 mg | 175 | Inventive |
| Sample 12 | 100 mg | 0 mg | 140 mg | 10 mg | 173 | Inventive |

Each group of 5 healthy adults was allowed to orally ingest one tablet of each of the samples 1 to 12 respectively within 30 minutes after meal every day, and this was repeated for 7 days. Feces were collected before commencement of the ingestion and on the next day of the final ingestion and stored in an anaerobic pack, and identification of bacteria by a culture test and measurement of ammonia quantity and pH were carried out within 30 hours.

Regarding detection of the intestinal flora, each bacterial group in the analytes was counted using a BS agar medium (for the genus Bifidobacterium), an NN agar medium (for the genus Clostridium) and a DHL agar medium (for the genus Enterobacter).

Average of each sample ingestion group is shown in Table 2. The number of cells and ammonia quantity are shown by relative values when the number of cells and quantity before ingestion are regarded as 100.

TABLE 2

| | | | | | Change in pH | |
|---|---|---|---|---|---|---|
| | | | | | before and | |
| | | | | Ammonia | after | |
| | Bifidobacterium | Clostridium | Enterobacter | (μg/g) | ingestion | Examples |
| Sample 1 | 99 | 100 | 102 | 105 | +0.1 | Comparative |
| Sample 2 | 105 | 95 | 90 | 93 | −0.1 | Comparative |
| Sample 3 | 35 | 97 | 104 | 110 | +0.2 | Comparative |
| Sample 4 | 128 | 82 | 89 | 89 | −0.2 | Inventive |
| Sample 5 | 195 | 8 | 46 | 60 | −0.6 | Inventive |
| Sample 6 | 120 | 60 | 64 | 82 | −0.4 | Inventive |
| Sample 7 | 220 | 15 | 42 | 53 | −0.5 | Inventive |
| Sample 8 | 340 | 0 | 21 | 46 | −1.0 | Inventive |
| Sample 9 | 348 | 0 | 5 | 40 | −1.6 | Inventive |
| Sample 10 | 312 | 0 | 25 | 48 | −0.9 | Inventive |
| Sample 11 | 188 | 9 | 53 | 72 | −0.5 | Inventive |
| Sample 12 | 190 | 11 | 52 | 75 | 0.0 | Inventive |

It was found that, by the ingestion of the samples of the invention, species of the genus Clostridium and species of the genus Enterobacter as intestinal toxic bacteria are significantly reduced, and species of the genus Bifidobacterium as good bacteria are increased. In addition, it was revealed that both of the pH of feces and ammonia quantity are significantly lowered to create an environment under which intestinal toxic bacteria are hard to live (generally, toxic bacteria easily propagate at around neutral pH).

Regarding the ingestion quantity of Salacia, the sample 5 showed a good result in comparison with the samples 4 and 6. It was considered that this is because three of the persons to be tested in the sample 6-ingestion group caused diarrhea.

In addition, the sample 9-ingestion group in which Salacia and catechin were concomitantly used changed to most favorable intestinal environment.

It was found that the intestinal toxic bacteria tend to increase by the ingestion of the Oolong tea powder alone, but it is suppressed by the concomitant use of Salacia and catechin.

### Example 2

Measurement of putrefaction products contained in the feces of before and after ingestion of the samples obtained in Example 1 was carried out using GC-9A manufactured by Shimadzu Corp.

Average of each sample ingestion group is shown in Table 3. Amounts of the putrefaction products, indole and skatole are shown by relative values when their amounts before ingestion are regarded as 100.

In addition, the questionnairing on the parsons to be tested was carried out regarding the before and after ingestion, and conditions of the skin and tiredness were scored based on the following criteria.

Conditions of the skin 5: Became good
4: Became slightly good
3: No change
2: Became slightly bad
1: Became bad

Tiredness 5: Became hard to tire
4: Became slightly hard to tire
3: No change
2: Became slightly easy to tire
1: Became easy to tire

Average values of the obtained scores are shown in Table 3.

TABLE 3

| | Putrefaction | Amount | | | | |
|---|---|---|---|---|---|---|
| | products | of | Amount of | Conditions | | |
| | Total | indole | skatole | of the | | |
| | (μg/g) | μg/g | μg/g | skin | Tiredness | Examples |
| Sample 1 | 102 | 110 | 102 | 2.8 | 3.2 | Comparative |
| Sample 2 | 101 | 96 | 95 | 3.2 | 3.0 | Comparative |
| Sample 3 | 123 | 131 | 210 | 2.0 | 2.2 | Comparative |
| Sample 4 | 75 | 81 | 82 | 3.8 | 3.8 | Inventive |
| Sample 5 | 55 | 62 | 50 | 4.2 | 4.0 | Inventive |
| Sample 6 | 62 | 69 | 66 | 4.0 | 3.8 | Inventive |
| Sample 7 | 46 | 42 | 14 | 4.4 | 4.2 | Inventive |
| Sample 8 | 30 | 35 | 4 | 4.6 | 4.4 | Inventive |
| Sample 9 | 22 | 11 | 0 | 4.6 | 4.6 | Inventive |
| Sample 10 | 32 | 38 | 22 | 4.6 | 4.2 | Inventive |
| Sample 11 | 60 | 62 | 54 | 4.0 | 4.2 | Inventive |
| Sample 12 | 63 | 52 | 44 | 4.6 | 4.0 | Inventive |

By the ingestion of the samples of the invention, amount of putrefaction products in the intestines were significantly lowered and conditions of the skin and tiredness were considerably improved.

In addition, particularly among the putrefaction products, reduction of the amount of indole and skatole was found.

Though ingestion of the lipase inhibitory material of the sample 3 increased putrefaction products in the intestines and worsened conditions of the skin, it was found that it becomes a proper state by changing into the constitutions of the invention which can be seen in the samples 10 to 12.

### Example 3

### Preparation of Tablets Using Salacia Extract Powder

By preparing tablets using the formulation shown in Table 4, a supplement to which shellac coating was applied was prepared.

TABLE 4
Tablet formulation example using the Salacia
extract powder of the invention

| Raw material name | Blending amount (wt %) |
|---|---|
| Salacia extract powder | 25.0 |
| Red wine polyphenol | 10.0 |
| Onion outer skin extract powder | 6.0 |
| Green tea extract | 15.0 |
| Hematococcus algal pigment | 1.0 |
| Chrome yeast | 4.0 |
| Carnitine | 10.0 |
| Crystalline cellulose | 23.0 |
| Sucrose fatty acid ester | 2.0 |
| Lactose | 1.0 |
| Calcium carbonate | 1.0 |
| Atomized silicon dioxide | 2.0 |

The effects shown by Examples 1 and 2 were obtained by the ingestion tablets of this formulation.

In addition, belly size became neat, the body became lighter, hangover became hard to occur and the like reports were obtained from the ingesting persons to be tested.

### INDUSTRIAL APPLICABILITY

An agent for reducing an intestinal toxic bacterium and a food article or pharmaceutical preparation to which its efficacy is applied are provided by the invention. Particularly, new effects of lowering intestinal pH, reducing intestinal ammonia concentration, reducing intestinal putrefaction product concentration, accelerating intestinal bifidobacterium propagation and improving chapped skin are provided.

The entire disclosure of each and every foreign patent application from which the benefit of foreign priority has been claimed in the present application is incorporated herein by reference, as if fully set forth.

1. An agent for reducing an intestinal toxic bacterium, which comprises:
a pulverized product or extract of a plant of the genus Salacia.

2. The agent for reducing an intestinal toxic bacterium according to claim 1, which shows an activity as a sucrase 50% inhibition concentration (IC₅₀ value) of 50 μg/ml or more and 1,000 μg/ml or less.

3. The agent for reducing an intestinal toxic bacterium according to claim 1, which further comprises:
from 1 to 50% by mass of catechin.

4. The agent for reducing an intestinal toxic bacterium according to claim 1, which further comprises:
from 2 to 80% by mass of polyphenols having lipase activity inhibitory effect.

5. The agent for reducing an intestinal toxic bacterium according to claim 1,
wherein the intestinal toxic bacterium to be reduced is a bacterium of the genus Enterobacter or a bacterium of the genus Clostridium.

6. The agent for reducing an intestinal toxic bacterium according to claim 1, which is an intestinal pH lowering agent.

7. The agent for reducing an intestinal toxic bacterium according to claim 1, which is an intestinal ammonia concentration reducing agent.

8. The agent for reducing an intestinal toxic bacterium according to claim 1, which is an intestinal putrefaction product concentration reducing agent.

9. The agent for reducing an intestinal toxic bacterium according to claim 8,
wherein the putrefaction product is indole or skatole.

10. The agent for reducing an intestinal toxic bacterium according to claim 1, which is an intestinal bifidobacterium propagation accelerator.

11. The agent for reducing an intestinal toxic bacterium according to claim 1, which is a chapped skin improving agent.

12. A food or drink or a food or drink material, which comprises:
the agent for reducing an intestinal toxic bacterium according to claim 1.

13. A tablet or hard capsule filling type food article or pharmaceutical preparation, which comprises:
the agent for reducing an intestinal toxic bacterium according to claim 1.
